(19) United States
(12) Patent Application Publication
(10) Pub. No.: US 2016/0122450 A1
(43) Pub. Date: May 5, 2016

(54) SYNTHESIS OF A SUBSTITUTED FURAN

(71) Applicant: Agency for Science, Technology and Research, Singapore (SG)

(72) Inventors: Yugen Zhang, Singapore (SG); Siew Ping Teong, Singapore (SG); Guangshun Yi, Singapore (SG)

(21) Appl. No.: 14/890,052

(22) PCT Filed: May 6, 2014
(86) PCT No.: PCT/SG2014/000198

§ 371 Date: Nov. 9, 2015

(30) Foreign Application Priority Data
May 8, 2013 (SG) 201303559-7

(51) Int. Cl. C08F 114/14; C07D 307/50

(52) U.S. Cl. CPC C08F 114/14; C07D 307/50

(57) ABSTRACT

The present invention provides a polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring. The present invention also provides a method for making an optionally substituted furan, the method comprising the step of subjecting a sugar to a dehydration reaction in the presence of a catalyst comprising said polymer.

### TECHNICAL FIELD

The present invention generally relates to a polymer and a method for converting a sugar to an optionally substituted furan in the presence of the polymer as a catalyst.

### BACKGROUND

Due to the increasing environmental concerns associated with the use of fossil fuels such as pollution and rapid depletion of sources, the development of alternative resources that are efficient and renewable has received significant attention. Recently, efforts have been directed towards developing reactions that convert renewable biomass resources to 5-hydroxyfurfural (HMF). HMF is a versatile and key intermediate in the biofuel and petrochemical industry. For example, HMF can be converted to 2,5-dimethylfuran which is a liquid biofuel that has a greater energy content than bioethanol. Oxidation of HMF also gives 2,5-furandicarboxylic acid, which has been proposed as a replacement for terephthalic acid in the production of polyesters. Other important chemicals HMF can be converted into include, but are not limited to, γ-valerolactone and 2,5-bis(hydroxymethyl)furan, which are both useful intermediates for the manufacture of alternative fuels, polyesters and polyurethanes.

However, the application of HMF generated from biomass is limited due to the inefficiency and high cost of its production. HMF generated from biomass is most commonly obtained by the dehydration of fructose. However, the overall efficiency of HMF production is hindered by multiple side reactions including rehydration of HMF to levulinic acid and formic acid, and condensation of HMF and fructose to form polymeric humins. The use of solvents such as dimethylsulfoxide (DMSO) and imidazolium ionic liquids (IL) are effective in suppressing undesirable side-reactions, but separating HMF from DMSO or IL is energy intensive due to the high boiling point of the solvents and the high solubility of HMF in these solvents. Consequently, the overall efficiency and cost-effectiveness of the process is low.

Alternatively, aqueous-organic biphasic solvent systems can be used for the reaction, but biphasic systems suffer from the drawback of having to use corrosive acid catalysts and inefficient extraction, which are both costly and harmful to the environment.

Several catalysts have been developed in an attempt to improve the efficiency and yield of the reactions. These include acidic resins, Zeolites, functionalised silica, functionalised MOF, heteropolyacids (HPA) and porous TiO₂/TiPO₄. However, these catalysts are not suitable for use in large scale production of HMF due to their low selectivity and stability. The catalysts are known to lose integrity of the catalytic site over time, leading to catalyst deactivation. In addition, the regeneration of these catalysts is difficult. Further, the abovementioned catalysts often require specific reaction conditions to be employed, making the industrial application of the reactions both difficult and costly. Moreover, the catalysts may also suffer from metal leaking issues that may cause profound environmental contamination.

There is therefore a need to provide a catalyst which may at least partially ameliorate one or more of the disadvantages described above.

### SUMMARY

In a first aspect, there is provided polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

Advantageously, the polymer comprises both an alkyl halide group and an ammonium halide group. These functional groups, present together, may be important to the advantageous properties of the polymer. Without being bound by theory, it is thought that these functional groups, when present together, may facilitate certain chemical reactions due to the presence of these functional groups. More advantageously, the presence of both the alkyl halide group and the optionally substituted ammonium halide group on the polymer may facilitate reactions that take advantage of the properties of both functionalities simultaneously. Advantageously, the alkyl halide groups may provide sites for hydrogen bonding and the ammonium halide groups may provide acidic sites for ion exchange or catalytic reactions. Advantageously, the polymer comprises both an alkyl halide group and an ammonium halide group, to enable the polymer to be used as a catalyst in a dehydration reaction of sugar.

Advantageously, the disclosed polymer may act as a weak acid in a dehydration reaction of a sugar as opposed to known catalysts which may be strong acids. Therefore, less harsh reaction conditions may be utilised when the disclosed polymer is used in a dehydration reaction of a sugar.

Advantageously, the aromatic rings may contribute to activating the alkyl halide and optionally substituted ammonium halide functional groups by modulating the electron density of these functional groups. This may further contribute to the polymer having advantageous properties such as facilitating ion-exchange and catalytic reactions. In a second aspect, there is provided a catalyst comprising a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

Advantageously, the polymer as disclosed above may be used as a catalyst. In an embodiment, the polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, may be used as a catalyst for converting a sugar to an optionally substituted furan.

In a third aspect, there is provided a method for making an optionally substituted furan, the method comprising the step of subjecting a sugar to a dehydration reaction in the presence of a catalyst comprising a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

In some embodiments, the sugar is fructose and the optionally substituted furan is 5-hydroxymethylfurfural (HMF). In some embodiments, the first aromatic ring type that has an alkyl halide group substitution on the aromatic ring is benzyl chloride and the second aromatic ring type that has an optionally substituted ammonium halide group substitution on the aromatic ring is optionally substituted benzylammonium chloride.

Advantageously, the disclosed method for making 5-hydroxymethylfurfural (HMF) may take advantage of the presence of both the first aromatic ring type that has an alkyl halide group substitution on the aromatic ring and the second aromatic ring type that has an optionally substituted ammonium halide group substitution on the aromatic ring in the catalyst. Advantageously, both the first aromatic ring type that has an alkyl halide group substitution on the aromatic ring and the second aromatic ring type that has an optionally substituted ammonium halide group substitution on the aromatic ring may be critical in facilitating the catalytic dehydration of the sugar. Both the benzyl chloride and the optionally substituted benzylammonium chloride may be critical in facilitating the catalytic dehydration of fructose.

Further advantageously, the optionally substituted ammonium chloride group may act as the acidic catalytic site. More advantageously, the plurality of alkyl halide groups may assist in binding the sugar to the catalytic surface of the catalyst, while the HCl may dissociated from the plurality of optionally substituted ammonium chloride groups under reaction condition to catalyse the dehydration reaction. Further advantageously, the plurality of benzyl chloride groups on the polymer surface may assist in binding the fructose to the polymer surface via H-bonding with functional groups of fructose, while the HCl dissociated from the plurality of optionally substituted benzylammonium chloride groups under reaction conditions may catalyse the dehydration reaction.

Advantageously, the disclosed method of making an optionally substituted furan may be significantly less harmful to living organisms and the environment. More advantageously, the disclosed method for synthesising an optionally substituted furan may utilise “green chemistry”. In some embodiments, the sugar is fructose and the optionally substituted furan is 5-hydroxymethylfurfural (HMF). Unlike traditional methods for synthesizing 5-hydroxymethylfurfural (HMF), the disclosed method may not use extremely toxic and/or poisonous reagents that may be harmful to organisms in the environment. More advantageously, the disclosed method may be safer, may not require the use of harsh and corrosive catalysts such as concentrated HCl or H₂SO₄, hence may not require special handling, may be more suitable for industrial scale synthesis and may be more sustainable. Advantageously, the method may use a catalyst that may act as a weak acid in a dehydration reaction of a sugar as opposed to known catalysts which may be a strong acid. Hence, less harsh reaction conditions may be utilised when the disclosed polymer is used in a dehydration reaction of a sugar.

More advantageously, due to the simplicity of the method, the disclosed method may be useful for industrial-scale synthesis of 5-hydroxymethylfurfural (HMF). The disclosed method may provide a simple and sustainable alternative for making HMF on an industrial scale. Advantageously, HMF may be a versatile and key intermediate in the biofuel and petrochemical industry. Therefore, a straight-forward and sustainable method for making HMF may lead to savings in resources and costs.

Further advantageously, the disclosed method for making 5-hydroxymethylfurfural (HMF) may proceed significantly more efficiently than conventional methods.

Advantageously, the reaction may proceed with high yield and high selectivity. More advantageously, the reaction may proceed in mild reaction conditions, under ambient temperature and pressure, in a variety of common and non-harmful solvents, may have a high conversion and yield and may not require high energy input for the reaction to proceed. Even further advantageously, since the reaction may proceed with high yield and limited by-products, it may be highly atomically efficient. Therefore, unlike conventional methods for synthesising HMF, the disclosed method may allow for efficient synthesis of HMF from fructose.

In a fourth aspect, there is provided a method for synthesizing a polymer, the method comprising, the step of mixing: (a) a starting material polymer comprising an aliphatic backbone having a plurality of aromatic rings that has an alkyl halide group substitution on the aromatic rings; and (b) an optionally substituted amine.

Advantageously, the disclosed method for synthesising the polymer may be straight-forward and sustainable. More advantageously, the disclosed method for synthesising the polymer may be performed at mild conditions and with environmentally benign reagents and solvents. Further advantageously, the method may allow the synthesis of polymers with different ratios of benzyl chloride to optionally substituted ammonium chloride groups.

In a fifth aspect, there is provided a method for reusing a catalyst comprising an aromatic ring substituted with an alkyl halide group and another aromatic ring substituted with an optionally substituted ammonium halide group, the method comprising the step of washing the catalyst with a solvent.

Advantageously, the catalyst may not become deactivated after repeated use. More advantageously, the catalyst may be reused without loss of activity. Further advantageously, the method for reusing the catalyst may be simple. Even further advantageously, the method for reusing the catalyst may not require catalyst regeneration. More advantageously, the catalyst may be reused multiple times following a simple step of washing with a solvent and drying.

Further advantageously, the reusing of the catalyst may be possible due to the equilibrium between the optionally substituted ammonium chloride and the amine/HCl in the catalyst. More advantageously, the catalyst may retain its acidic active sites during the reaction cycle, enabling repeated reuse without suffering from decreased activity over multiple use and catalyst deactivation.

### DEFINITIONS

The following words and terms used herein shall have the meaning indicated:

The term “green chemistry” and “sustainable chemistry” may be used interchangeably, and refer to a philosophy of chemical research and engineering that encourages the design of products and processes that minimize the use and generation of substances that are harmful to organisms in the environment. The terms “green” and “sustainable” for the purposes of this disclosure, should be construed accordingly.

The terms “recyclable” and “reusable” in the context of this disclosure, may be used interchangeably, and refer to the ability to restore the catalytic activity of the catalyst after repeated and/or extended use. The terms “recyclability”, “recycling”, “reusability” and “reusing” should be construed accordingly.

The term ‘alkyl’, as a group or part of a group, may be a straight or branched aliphatic hydrocarbon group. The alkyl may be a C₁-C₂₀ alkyl group. The alkyl may be a C₁-C₁₀ alkyl group. Straight and branched C₁-C₁₀ alkyl substituents may be selected from the group consisting of methyl, ethyl, propyl, butyl, pentyl, hexyl, heptyl, octyl, nonyl, decyl and any isomers thereof. The alkyl may be selected from the group consisting of methyl, n-ethyl, n-propyl, 2-propyl, n-butyl, sec-butyl, iso-butyl, tert-butyl, n-pentyl, 3-methyl-1-butyl, 2-methyl-1-butyl, 2,2,-dimethyl-1-propyl, 3-pentyl, 2-pentyl, 3-methyl-2-butyl and 2-methyl-2-butyl. The alkyl may be a terminal group or a bridging group.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, isocyanate, —C(O)NH(alkyl), and —C(O)N(alkyl)2.

The term “aromatic ring”, as used herein, refers to monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like. The term also encompasses “heteroaromatic rings” and variants such as “heteroaryl” or “heteroarylene”, which include monovalent (“heteroaryl”) and divalent (“heteroarylene”), single, polynuclear, conjugated and fused aromatic radicals having 6 to 20 atoms wherein 1 to 6 atoms are heteroatoms selected from O, N, NH and S. Examples of such groups include pyridyl, 2,2′-bipyridyl, phenanthrolinyl, quinolinyl, thiophenyl, and the like.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the terms “about” and “approximately”, in the context of concentrations of components of the formulations, or where applicable, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Optional Embodiments

A polymer may comprise an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

The polymer may have the number ratio of first aromatic ring types represented as “n” to second aromatic ring types represented as “m” selected to catalyse the dehydration of a sugar.

The polymer may have the number ratio of n:m in the range that may be optimal for the catalytic dehydration of a sugar. The polymer may have the number ratio of n:m in the range of about 20:1 to about 1:20, about 10:1 to about 1:20, about 5:1 to about 1:20, about 2:1 to about 1:20, about 1:1 to about 1:20, about 1:2 to about 1:20, about 1:5 to about 1:20, about 1:10 to about 1:20, about 20:1 to about 1:10, about 10:1 to about 1:10, about 5:1 to about 1:10, about 2:1 to about 1:10, about 1:1 to about 1:10, about 1:2 to about 1:10, about 1:5 to about 1:10, about 20:1 to about 1:5, about 10:1 to about 1:5, about 5:1 to about 1:5, about 2:1 to about 1:5, about 1:1 to about 1:5, about 1:2 to about 1:5, about 20:1 to about 1:2, about 10:1 to about 1:2, about 5:1 to about 1:2, about 2:1 to about 1:2, about 1:1 to about 1:2, about 20:1 to about 1:1, about 10:1 to about 1:1, about 5:1 to about 1:1, about 2:1 to about 1:1, about 20:1 to about 2:1, about 15:1 to about 2:1, about 10:1 to about 2:1, about 5:1 to about 2:1, about 20:1 to about 5:1, about 10:1 to about 5:1 or about 20:1 to about 10:1. The number ratio of n:m may be in the range of about 10:1 to about 1:10.

The aliphatic backbone may be an optionally substituted straight chain aliphatic polymer. The aliphatic backbone may be an optionally substituted branched aliphatic polymer. The aliphatic backbone may be a homopolymer. The aliphatic backbone may be a copolymer. The aliphatic backbone may be saturated or unsaturated. The aliphatic backbone may be a saturated optionally substituted straight chain aliphatic polymer. The aliphatic backbone may be an alkane chain.

The aromatic ring may be substituted or unsubstituted. The aromatic ring may comprise a conjugated planar ring system. The aromatic ring may comprise only of carbon ring atoms. The aromatic ring may be heteroaromatic. The aromatic ring may comprise non-carbon ring atoms. The non-carbon ring atoms may be selected, from the group consisting of oxygen, nitrogen and sulfur. The aromatic ring may be monocyclic, bicyclic or polycylic. The aromatic ring may be benzene, naphthalene or anthracene. A monocyclic aromatic ring may be a 3-membered, 4-membered, 5-membered, 6-membered, 7-membered or an 8-membered ring. A monocyclic aromatic ring may be a 5-membered or a 6-membered ring. Bicyclic or polycylic aromatic rings may comprise monocyclic aromatic rings that share connecting bonds. The aromatic ring may be benzene.

The alkyl of the alkyl halide group of the first aromatic ring type may be selected from the group consisting of methyl, optionally substituted ethyl, optionally substituted propyl, optionally substituted butyl, optionally substituted pentyl and any isomers thereof. The alkyl of the alkyl halide group of the first aromatic ring type may be selected from the group consisting of methyl, ethyl, propyl, butyl, pentyl, hexyl, heptyl, octyl, nonyl, decyl and any isomers thereof. The alkyl may be selected from the group consisting of methyl, n-ethyl, n-propyl, 2-propyl, n-butyl, sec-butyl, iso-butyl, tert-butyl, n-pentyl, 3-methyl-1-butyl, 2-methyl-1-butyl, 2,2,-dimethyl-1-propyl, 3-pentyl, 2-pentyl, 3-methyl-2-butyl and 2-methyl-2-butyl.

The halide of the alkyl halide group of the first aromatic ring type may be selected from the group consisting of fluoride, chloride, bromide and iodide.

The alkyl halide group of the first aromatic ring type may be selected from the group consisting of methyl halide, ethyl halide, propyl halide, n-propyl halide, butyl halide, n-butyl halide, pentyl halide, n-pentyl halide. The alkyl halide group of the first aromatic ring type may be selected from the group consisting of alkyl fluoride, alkyl chloride, alkyl bromide and alkyl iodide. The methyl halide may be methyl fluoride, methyl chloride, methyl bromide or methyl iodide. The alkyl chloride may be methyl chloride, ethyl chloride, n-ethyl chloride, propyl chloride, n-propyl chloride, butyl chloride, n-butyl chloride, pentyl chloride or n-pentyl chloride. The alkyl halide group of the first aromatic ring type may be methyl chloride.

The first aromatic ring type that has an alkyl halide group substitution on the aromatic ring may be fluorobenzene, chlorobenzene, bromobenzene or iodobenzene. The first aromatic ring type that has an alkyl halide group substitution on the aromatic ring may be benzyl fluoride, benzyl chloride, benzyl bromide or benzyl iodide. The first aromatic ring type that has an alkyl halide group substitution on the aromatic ring may be (1-fluoroethyl)benzene, (1-chloroethyl)benzene, (1-bromoethyl)benzene or (1-iodoethyl)benzene. The first aromatic ring type that has an alkyl halide group substitution on the aromatic ring may be (1-chloromethyl)benzene, (1-chloroethyl)benzene, (1-chloropropyl)benzene, (2-chloropropyl)benzene, (1-chlorobutyl)benzene, (1-chloro-2-methylpropyl)benzene, (1-chloro-1-methylpropyl)benzene, (1-chloro-1,1-dimethylethyl)benzene, (1-chloropentyl)benzene, (1-chloro-3-methylbutyl)benzene), (1-chloro-2-methylbutyl)benzene, (1-chloro-2,2-dimethylpropyl)benzene, (1-chloro-3-pentyl)benzene, (1-chloro-2-pentyl)benzene or (1-chloro-2-methyl-2-butyl)benzene.

The aromatic ring of the first aromatic ring type may be substituted at the o-position, the m-position or the p-position.

The first aromatic ring type that has an alkyl halide group substitution on the aromatic ring may be benzyl chloride. The benzyl chloride may be o-benzyl chloride, m-benzyl chloride or p-benzyl chloride.

The optionally substituted ammonium of the optionally substituted ammonium chloride group of the second aromatic ring type may be an optionally substituted ammonium ion. The optionally substituted ammonium may be an aminium ion. The optionally substituted ammonium may be cationic. The optionally substituted ammonium may be selected from the group consisting of primary ammonium, secondary ammonium, tertiary ammonium and quaternary ammonium depending on the number of substituents. The optionally substituted ammonium may be substituted with alkyl groups. The primary, secondary and tertiary ammonium ions may be Brønsted acids. The primary, secondary and tertiary ammonium ions may be weak acids.

The optionally substituted ammonium halide group of the second aromatic ring type may be optionally substituted ammonium fluoride, optionally substituted ammonium chloride, optionally substituted ammonium bromide or optionally substituted ammonium iodide. The optionally substituted ammonium halide may be optionally substituted ammonium chloride.

The second aromatic ring type that has an optionally substituted ammonium halide group substitution on the aromatic ring may be optionally substituted anilinium fluoride, optionally substituted anilinium chloride, optionally substituted anilinium bromide or optionally substituted anilinium iodide. The second aromatic ring type that has an optionally substituted ammonium halide group substitution on the aromatic ring may be optionally substituted benzyl ammonium fluoride, optionally substituted benzylammonium chloride, optionally substituted benzylammonium bromide or optionally substituted benzylammonium iodide. The second aromatic ring type that has an optionally substituted ammonium halide group substitution on the aromatic ring may be optionally substituted phenylethylammonium fluoride, optionally substituted phenylethylammonium chloride, optionally substituted phenylethylammonium bromide or optionally substituted phenylethylammonium iodide. The second aromatic ring type that has an optionally substituted ammonium halide group substitution on the aromatic ring may be optionally substituted phenylmethylammonium chloride, optionally substituted phentyl-1-ethylammonium chloride, optionally substituted phenyl-2-ethylammonium chloride, optionally substituted phenyl-1-propylammonium chloride, optionally substituted phenyl-2-propylammonium chloride, optionally substituted phenyl-1-butylammonium chloride, optionally substituted phenyl-2-methylpropylammonium chloride, optionally substituted phenyl-1-methylpropylammonium chloride, optionally substituted phenyl-1,1-dimethylethylammonium chloride, optionally substituted phenyl-2-pentylammonium chloride, optionally substituted phenyl-3-methylbutylammonium chloride, optionally substituted phenyl-2-methylbutylamminium chloride, optionally substituted phenyl-2,2-dimethylpropylammonium chloride, optionally substituted phenyl-3-pentylammonium chloride, optionally substituted phenyl-2-pentylammonium chloride and optionally substituted phenyl-2-methyl-2-butylammonium chloride.

The aromatic ring of the second aromatic ring type may be substituted at the o-position, the m-position or the p-position.

The second aromatic ring type that has an optionally substituted ammonium halide group substitution on the aromatic ring may be optionally substituted benzylammonium chloride. The optionally substituted benzylammonium chloride may be optionally substituted o-benzylammonium chloride, optionally substituted m-benzylammonium chloride or optionally substituted p-benzylammonium chloride.

The optionally substituted benzylammonium chloride may be selected from the group consisting of benzylammonium chloride, benzylmethylammonium chloride, benzyldimethylammonium chloride, benzyltrimethylammonium chloride, benzylethylammonium chloride, benzyldiethylammonium chloride, benzyltriethylammonium chloride, benzylpropylammonium chloride, benzyldipropylammonium chloride, benzyltripropylammonium chloride, benzylmethylethylammonium chloride, benzylmethylpropylammonium chloride, benzylethylpropylammonium chloride, benzyldimethylethylammonium chloride, benzyldiethylmethylammonium chloride, benzyldimethylpropylammonium chloride, benzyldipropylmethylammonium chloride, benzyldiethylpropylammonium chloride, benzyldipropylethylammonium chloride, methylethylpropylammonium chloride and benzylurea chloride.

The optionally substituted benzylammonium chloride may be selected from the group consisting of o-benzylammonium chloride, m-benzylammonium chloride, p-benzylammonium chloride, o-benzylurea chloride, m-benzylurea chloride, p-benzylurea chloride, o-diethylbenzylammonium chloride, m-diethylbenzylammonium chloride and p-diethylbenzylammonium chloride.

A catalyst may comprise a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

A polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, may be a catalyst.

A polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, may be used as a catalyst.

A polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, may be a catalytic polymer.

A polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, may be used as a catalyst for converting a sugar to an optionally substituted furan.

A method for making an optionally substituted furan may comprise the step of subjecting a sugar to a dehydration reaction in the presence of a catalyst comprising a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

The sugar may be short-chain carbohydrate. The sugar may be a monosaccharide. The sugar may be a disaccharide. The sugar may be a polysaccharide. The sugar may have a chemical formula Cₓ(H₂O)_{y}, where x≧3. The monosaccharide may be a diose, triose, pentose, hexose or heptose. The monosaccharide may be an aldose or a ketose. The monosaccharide may be glycerose, erythrose, threose, ribose, xylose, allose, glucose, dextrose, fructose, levulose, galactose, gulose or idose. The monosaccharide may be D-glycerose, D-erythrose, D-threose, D-ribose, D-xylose, D-allose, D-glucose, D-dextrose, D-fructose, D-levulose, D-galactose, D-gulose or D-idose. The monosaccharide may be L-glycerose, L-erythrose, L-threose, L-ribose, L-xylose, L-allose, L-glucose, L-dextrose, L-fructose, L-levulose, L-galactose, L-gulose or L-idose. The monosaccharide may be fructose. The monosaccharide may be D-fructose. The monosaccharide may be L-fructose.

The optionally substituted furan may comprise an aldehyde and an alcohol. The furan may be a 5-membered aromatic ring with four carbon atoms and one oxygen. The aldehyde substituent may be an alkanal. The alkanal may be methanal, optionally substituted ethanal, optionally substituted propanal, optionally substituted butanal, optionally substituted pentanal and any isomers thereof. The alcohol may be an alkanol. The alkanol may be methanol, optionally substituted ethanol, optionally substituted propanol, optionally substituted butanol, optionally substituted pentanol and any isomers thereof. The alkanol may be hydroxyl, optionally substituted hydroxymethyl, optionally substituted hydroxyethyl, optionally substituted hydroxypropyl, optionally substituted hydroxybutyl and any isomers thereof.

The furan may be substituted at the 2-position, 3-position, 4-position or 5-position of the furan. The furan may be substituted at the 2-potion and the 5-position. The furan may be substituted with an alkanal at the 2-potion. The furan may be substituted with an alkanol at the 5-position. The furan may be substituted with an alkanal at the 2-position and an alkanol at the 5-position. The furan may be substituted with an ethanal at the 2-position and ethanol at the 5-position.

The optionally substituted furan may be an optionally substituted furfural.

The optionally substituted furan may be 5-(hydroxymethyl)-2-furaldehyde. The optionally substituted furan may be 5-hydroxymethylfurfural (HMF).

Both the first aromatic ring type that has an alkyl halide group substitution on the aromatic ring and the second aromatic ring type that has an optionally substituted ammonium halide group substitution on the aromatic ring may be important in facilitating the catalytic dehydration of the sugar. Both the benzyl chloride groups and the optionally substituted ammonium chloride groups may be important in facilitating the catalytic dehydration of fructose.

The plurality of alkyl halide groups may assist in binding the sugar to the catalytic surface of the catalyst, while the HCl may dissociated from the plurality of optionally substituted ammonium chloride groups under reaction condition to catalyse the dehydration reaction. The plurality of benzyl chloride groups may assist in binding the fructose to the catalytic surface via H-bonding with functional groups of fructose, while the HCl dissociated from the plurality of optionally substituted ammonium chloride groups may catalyse the dehydration reaction.

The dissociation of HCl from the optionally substituted ammonium chloride group at elevated temperatures may be the origin of catalytic activity. The dissociated HCl may catalyse the dehydration reaction to convert a sugar to an optionally substituted furan. The optionally substituted ammonium chloride group may act as the acidic catalytic site.

The dehydration reaction may comprise the loss of a water molecule. Because the hydroxyl group (—OH) is a poor leaving group, having a Brønsted acid catalyst may help by protonating the hydroxyl group to give the better leaving group, —OH₂⁺. Common dehydrating agents used in organic synthesis may be concentrated sulfuric acid, concentrated phosphoric acid, hot aluminium oxide or hot ceramic.

The dehydration reaction may be performed at a temperature in the range of about 80° C. to about 220° C., about 80° C. to about 100° C., about 80° C. to about 120° C., about 80° C. to about 140° C., about 80° C. to about 160° C., about 80° C. to about 180° C., about 80° C. to about 200° C., about 100° C. to about 120° C., about 100° C. to about 140° C., about 100° C. to about 160° C., about 100° C. to about 180° C., about 100° C. to about 200° C., about 100° C. to about 220° C., about 120° C. to about 140° C., about 120° C. to about 160° C., about 120° C. to about 180° C., about 120° C. to about 200° C., about 120° C. to about 220° C., about 140° C. to about 160° C., about 140° C. to about 180° C., about 140° C. to about 200° C., about 140° C. to about 220° C., about 160° C. to about 180° C., about 160° C. to about 200° C., about 160° C. to about 220° C., about 180° C. to about 200° C., about 180° C. to about 220° C. or about 200° C. to about 220° C. The dehydration reaction may be performed at a temperature in the range of about 120° C. to about 180° C. Performing the reaction at temperatures in this range may cause the dissociation of HCl from the optionally substituted ammonium chloride group in the catalyst. The dissociated HCl may catalyse the dehydration reaction.

The dehydration reaction may be performed at atmospheric pressure. The dehydration reaction may be performed at pressures above 1 atm.

The dehydration reaction may be performed for a time period in the range of about 0.1 hours to about 20 hours, about 0.1 hours to about 0.5 hours, about 0.1 hours to about 1 hours, about 0.1 hours to about 2 hours, about 0.1 hours to about 5 hours, about 0.1 hours to about 10 hours, about 0.5 hours to about 1 hours, about 0.5 hours to about 2 hours, about 0.5 hours to about 5 hours, about 0.5 hours to about 10 hours, about 0.5 hours to about 20 hours, about 1 hour to about 2 hours, about 1 hour to about 5 hours, about 1 hour to about 10 hours, about 1 hour to about 20 hours, about 5 hours to about 10 hours, about 5 hours to about 20 hours or about 10 hours to about 20 hours.

The catalyst may be present in an amount in the range of about 0.1 mol % to about 30 mol % based on optionally substituted ammonium chloride to fructose, about 0.1 mol % to about 1 mol % based on optionally substituted ammonium chloride to fructose, about 0.1 mol % to about 2 mol % based on optionally substituted ammonium chloride to fructose, about 0.1 mol % to about 5 mold based on optionally substituted ammonium chloride to fructose, about 0.1 mol % to about 10 mol % based on optionally substituted ammonium chloride to fructose, about 0.1 mol % to about 20 mol % based on optionally substituted ammonium chloride to fructose, 1 mol % to about 2 mol % based on optionally substituted ammonium chloride to fructose, about 1 mol % to about 5 mol % based on optionally substituted ammonium chloride to fructose, about 1 mol % to about 10 mol % based on optionally substituted ammonium chloride to fructose, about 1 mol % to about 20 mold based on optionally substituted ammonium chloride to fructose, about 1 mol % to about 30 mol % based on optionally substituted ammonium chloride to fructose, 2 mol % to about 5 mol % based on optionally substituted ammonium chloride to fructose, about 2 mol % to about 10 mol % based on optionally substituted ammonium chloride to fructose, about 2 mol % to about 20 mol % based on optionally substituted ammonium chloride to fructose, about 2 mol % to about 30 mol % based on optionally substituted ammonium chloride to fructose, about 5 mol % to about 10 mol % based on optionally substituted ammonium chloride to fructose, about 5 mol % to about 20 mol % based on optionally substituted ammonium chloride to fructose, about 5 mol % to about 30 mol % based on optionally substituted ammonium chloride to fructose, 10 mol % to about 20 mol % based on optionally substituted ammonium chloride to fructose, about 10 mol % to about 30 mol % based on optionally substituted ammonium chloride to fructose or about 20 mol % to about 30 mol % based on optionally substituted ammonium chloride to fructose. The catalyst may be present in an amount in the range of 10 mol % to 20 mol % based on optionally substituted ammonium chloride to fructose.

The catalyst may be present in an amount in the range of about 0.1 mol % to about 30 mol % based on total chloride to fructose, about 0.1 mol % to about 1 mol % based on total chloride to fructose, about 0.1 mol % to about 2 mol % based on total chloride to fructose, about 0.1 mol % to about 5 mol % based on total chloride to fructose, about 0.1 mol % to about 10 mol % based on total chloride to fructose, about 0.1 mol % to about 20 mol % based on total chloride to fructose, 1 mol % to about 2 mol % based on total chloride to fructose, about 1 mol % to about 5 mol % based on total chloride to fructose, about 1 mol % to about 10 mol % based on total chloride to fructose, about 1 mol % to about 20 mol % based on total chloride to fructose, about 1 mol % to about 30 mol % based on total chloride to fructose, 2 mol % to about 5 mol % based on total chloride to fructose, about 2 mol % to about 10 mol % based on total chloride to fructose, about 2 mol % to about 20 mol % based on total chloride to fructose, about 2 mol % to about 30 mol % based on total chloride to fructose, about 5 mol % to about 10 mol % based on total chloride to fructose, about 5 mol % to about 20 mol % based on total chloride to fructose, about 5 mol % to about 30 mol % based on total chloride to fructose, 10 mol % to about 20 mol % based on total chloride to fructose, about 10 mol % to about 30 mol % based on total chloride to fructose or about 20 mol % to about 30 mol % based on total chloride to fructose. The catalyst may be present in an amount in the range of 10 mol % to 20 mol % based on total chloride to fructose.

The catalyst may be present in an amount in the range of about 0.1 mol % to about 30 mol % based on benzyl chloride to fructose, about 0.1 mol % to about 1 mol % based on benzyl chloride to fructose, about 0.1 mol % to about 2 mol % based on benzyl chloride to fructose, about 0.1 mol % to about 5 mol % based on benzyl chloride to fructose, about 0.1 mol % to about 10 mol % based on benzyl chloride to fructose, about 0.1 mol % to about 20 mol % based on benzyl chloride to fructose, about 1 mol % to about 2 mol % based on benzyl chloride to fructose, about 1 mol % to about 5 mol % based on benzyl chloride to fructose, about 1 mol % to about 10 mol % based on benzyl chloride to fructose, about 1 mol % to about 20 mol % based on benzyl chloride to fructose, about 1 mol % to about 30 mol % based on benzyl chloride to fructose, 2 mol % to about 5 mol % based on benzyl chloride to fructose, about 2 mol % to about 10 mol % based on benzyl chloride to fructose, about 2 mol % to about 20 mol % based on benzyl chloride to fructose, about 2 mol % to about 30 mol % based on benzyl chloride to fructose, about 5 mol % to about 10 mol % based on benzyl chloride to fructose, about 5 mol % to about 20 mol % based on benzyl chloride to fructose, about 5 mol % to about 30 mol % based on benzyl chloride to fructose, 10 mol % to about 20 mol % based on benzyl chloride to fructose, about 10 mol % to about 30 mol % based on benzyl chloride to fructose or about 20 mol % to about 30 mol % based on benzyl chloride to fructose. The catalyst may be present in an amount in the range of 10 mol % to 20 mol % based on benzyl chloride to fructose.

The dehydration reaction may further comprise a solvent. The solvent may be aqueous or organic. The solvent may be protic or aprotic. The solvent may be polar or non-polar. The solvent may be a mixture of solvents. The mixture of solvents may be miscible or immiscible. The solvent may be mono-phasic or bi-phasic. The solvent may be selected from the group consisting of water, isopropanol, 1-butanol, N,N-dimethylformamide (DMF), dimethylsulfoxide (DMSO), acetone, acetonitrile, tetrahydrofuran (THF) and any mixtures thereof. The solvent may be isopropanol. Isopropanol may be less toxic. The isopropranol may be reuseable.

A method for making 5-hydroxymethylfurfural may comprise the step of subjecting a sugar to a dehydration reaction in the presence of a catalyst comprising a polymer having an aliphatic backbone having a plurality of aromatic rings bonded thereon, said plurality of aromatic rings comprising benzyl chloride and optionally substituted benzylammonium chloride.

A method for making 5-hydroxymethylfurfural may comprise the step of subjecting a sugar to a dehydration reaction in the presence of a catalyst comprising a polymer having an aliphatic backbone having a plurality of aromatic rings bonded thereon, said plurality of aromatic rings comprising benzyl chloride and benzylammonium chloride.

A method for synthesizing a polymer, the method comprising the step of mixing: (a) a starting material polymer comprising an aliphatic backbone having a plurality of aromatic rings that has an alkyl halide group substitution on the aromatic rings; and (b) an optionally substituted amine.

The starting material polymer comprising an aliphatic backbone having a plurality of aromatic rings that has an alkyl halide group substitution on the aromatic rings may be poly(vinylbenzyl fluoride), poly(vinylbenzyl chloride), poly(vinylbenzyl bromide) or poly(vinylbenzyl iodide).

The optionally substituted amine may be ammonia, a primary amine, secondary amine, tertiary amine or urea. The primary amine may be methylamine, n-ethylamine, n-propylamine, 2-propylamine, n-butylamine, sec-butylamine, iso-butylamine, tert-butylamine, n-pentylamine, 3-methyl-1-butylamine, 2-methyl-1-butylamine, 2,2-dimethyl-1-propylamine, 3-pentylamine, 2-pentylamine, 3-methyl-2-butylamine or 2-methyl-2-butylamine. The secondary amine may be dimethylamine, diethylamine, dipropylamine, dibutylamine, dipentylamine, methylethylamine, methylpropylamine or ethylpropylamine. The tertiary amine may be trimethylamine, triethylamine, tripropylamine, tributylamine, tripentylamine, dimethylethylamine, diethylmethylamine, dimethylpropylamine, dipropylmethylamine, diethylpropylamine, dipropylethylamine or methylethylpropylamine. The optionally substituted amine may be selected from the group consisting of ammonia, urea and diethylamine.

The optionally substituted amine may be mixed with the poly(vinylbenzyl chloride) at a chloride:amine mole ratio in the range of about 1:0.5 to about 1:150, about 1:1 to about 1:150, about 1:10 to about 1:150, about 1:50 to about 1:150, about 1:100 to about 1:150, about 1:0.5 to about 1:100, about 1:1 to about 1:100, about 1:10 to about 1:100, about 1:50 to about 1:100, about 1:0.5 to about 1:50, about 1:1 to about 1:50, about 1:10 to about 1:50, about 1:0.5 to about 1:10, about 1:1 to 1:10 or about 1:0.5 to about 1:1.

The mixing step may be performed at a temperature in the range of about 50° C. to about 150° C., about 50° C. to about 75° C., about 50° C. to about 100° C., about 50° C. to about 125° C., about 75° C. to about 100° C., about 75° C. to about 125° C., about 75° C. to about 150° C., about 100° C. to about 125° C., about 100° C. to about 150° C. or about 120° C. to about 150° C.

The mixing step may be performed at atmospheric pressure.

The method for synthesizing the polymer may further comprise the step of subjecting the polymer to dilute HCl. The concentration of the HCl may be in the range of about 0.1 M to 1 M. The dilute HCl may protonate the polymer to form the optionally substituted ammonium halide groups.

A method for reusing a catalyst comprising a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, the method comprising the step of washing the catalyst with a solvent.

The catalyst comprising a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring, may be reused. The method for reusing the catalyst may comprise washing the catalyst with a solvent. The solvent may be aqueous or organic. The solvent may be protic or aprotic. The solvent may be polar or non-polar. The solvent may be selected from the group consisting of water, isopropanol, 1-butanol, N,N-dimethylformamide (DMF), dimethylsulfoxide (DMSO), acetone, acetonitrile and any mixtures thereof. The method for reusing may comprise washing the catalyst with methanol.

The method for reusing the catalyst may further comprise drying the catalyst under vacuum. The drying may be performed at a temperature in the range of about 20° C. to about 80° C. The drying may be performed for a time period in the range of about 0.5 hours to about 4 hours.

The reusing of the catalyst may be possible due to the equilibrium between the optionally substituted ammonium chloride and the amine/HCl in the catalyst. The catalyst may retain its acidic active sites during multiple reaction cycles, enabling repeated reuse without suffering from decreased activity over multiple use and catalyst deactivation.

### BRIEF DESCRIPTION OF DRAWINGS

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 is a diagram showing the roles of benzyl chloride and ammonium chloride in the catalytic dehydration of fructose.

FIG. 2(a) is a graph showing the effect of catalyst loading on the kinetics of the dehydration reaction of fructose.

FIG. 2(b) is a graph showing the effect of reaction temperature on the kinetics of the dehydration reaction of fructose.

FIG. 3 is a graph showing the yield of HMF using the same catalyst in 10 rounds of reactions.

### EXAMPLES

Non-limiting examples of the invention will be further described in greater detail by reference to specific Examples, which should not be construed as in any way limiting the scope of the invention. Based on the foregoing disclosure, it should be clear that by the method, the objectives set forth herein can be fulfilled.

### Example 1

### Preliminary Testing

Several Brønsted acids were tested for their ability to catalytically convert fructose to 5-hydroxymethylfurfural (HMF) in isopropanol. Table 1 summarizes the catalysts that were screened and the yield of HMF obtained when fructose (180 mg) and the Brønsted acid (10 mol %) were stirred in isopropanol (2 mL) at 120° C. for 2 hours. For the reaction using HCl as the Brønsted acid, the yield is the NMR yield. Strong acids, which are known to catalyse the dehydration reaction of fructose to form HMF, but are not suitable for sustainable industrial scale synthesis of HMF due to problems such as corrosiveness and difficulty in handling as well as catalyst deactivation, were also included for comparison.

TABLE 1
Catalyst screening for production of HMF from fructose
in isopropanol.

| Bronsted acid | Yield (%) |
|---|---|
| HCl | 82 |
| H₂SO₄ | 68 |
| HNO₃ | <5 |
| H₃PO₄ | 0 |
| HCOOH | 0 |
| CH₃COOH | 0 |
| B(OH)₃ | 0 |
| NH₄Cl | 60 |
| 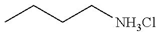 | 22 |
| 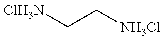 | 65 |
| 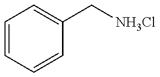 | 72 (2 hours) 80 (5 hours) |
| 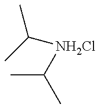 | <5 |

As shown in Table 1, the catalytic activities of the strong acids were found to be proportional to the Brønsted acidity. However, the weakly acidic ammonium salts did not follow this expected trend. For example, benzylammonium chloride salts gave a very high yield of HMF (up to 80% in 5 hours). This was the first time ammonium salts were demonstrated to be efficient catalysts for converting fructose to HMF.

The dissociation of HCl from the ammonium chloride group at elevated temperatures is believed to be the origin of catalytic activity. The equilibrium between the ammonium chloride salt and the HCl/amine makes the compound reusable as a catalyst.

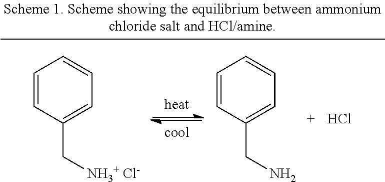

### Example 2

### The Catalytic Polymers

The high activity of ammonium salt catalysts, as tested in Example 2, lead to the development of a polystyrene based poly-benzylic ammonium salt polymer as a novel heterogeneous catalyst suitable for use in the industrial scale production of HMF. The ammonium polymers were synthesized from poly(vinylbenzyl chloride) (P-BnCl) polymers with various sources of ammonia. Polymers with different ammonium loadings were synthesized and tested for their ability to catalyse fructose dehydration to HMF.

### Materials and Instruments:

All solvents and chemicals were used as obtained from commercial suppliers, unless otherwise indicated. NMR spectra were recorded on a Bruker AV-400 (400 MHz). Progress of the reaction (conversion) was typically monitored by the SU-300 Sugar Analyzer (TOA-DKK Corp.). The loading of the polymers was calculated using elemental analysis. Poly-benzyl chloride (P-BnCl) was purchased from Aldrich (Product No.: 63868).

### Preparation of Polymer A:

Poly(vinylbenzyl chloride) (P-BnCl) polymer (1 g, 5.5 mmol Cl/g, 2.75 mmol) and aq. NH₃ (4.68 mmol) was stirred in DMF (15 mL) at 80° C. for 24 hours. The polymer was washed with DMF (15 mL×5), DMF/H₂O (1:1 v/v, 15 mL×5), MeOH (10 mL×5), filtered and dried under vacuum at 50° C. for 24 hours. After synthesis, the catalysts were stirred in dilute HCl solution (5M, 28 mL) for 1 hour, washed with MeOH, filtered and dried under vacuum at 50° C. for 6 hours.

### Preparation of Polymer B:

Poly(vinylbenzyl chloride) (P-BnCl) polymer (1 g, 5.5 mmol Cl/g, 2.75 mmol), urea (1.67 g, 27.8 mmol) and H₂O (1 mL, 55.5 mmol) were stirred in CH₃CN (20 mL) at 120° C. for 48 hours. The polymer was then washed with H₂O, acetone, filtered and dried under vacuum at 50° C. for 24 hours. The resultant polymer was stirred in dilute HCl solution (5M, 28 mL) for 1 hour, washed with methanol and dried under vacuum at 50° C. for 6 hours.

### Preparation of Polymer C:

Poly(vinylbenzyl chloride) (P-BnCl) polymer (1 g, 5.5 mmol Cl/g, 2.75 mmol) and aqueous NH₃ (4.68 mmol) were stirred in DMF (15 mL) at 80° C. for 24 hours. The polymer was washed with DMF (15 mL×5), DMF/H2O (1:1 v/v, 15 mL×5), MeOH (10 mL×5), filtered and dried under vacuum at 50° C. for 24 hours. The resultant polymer was stirred in dilute HCl solution (5M, 28 mL) for 1 hour, washed with methanol and dried under vacuum at 50° C. for 6 hours.

### Preparation of Polymer D:

poly(vinylbenzyl chloride) (P-BnCl) polymer (1 g, 5.5 mmol Cl/g, 2.75 mmol) and urea (5 mmol) and water (10 mmol) were stirred in acetonitrile (33 mL) at 120° C. for 48 hours. The polymer was washed with water, acetone, filtered and dried under vacuum at 50° C. for 6 hours. The resultant polymer was stirred in dilute HCl solution (5M, 28 mL) for 1 hour, washed with methanol and dried under vacuum at 50° C. for 6 hours.

### Preparation of Polymer E:

Poly(vinylbenzyl chloride) (P-BnCl) polymer (1 g, 5.5 mmol Cl/g, 2.75 mmol) and aqueous NH₃ (600 mmol) were stirred in DMF (15 mL) at 120° C. for 48 hours. The polymer was washed with water and acetone, then filtered and dried under vacuum at 50° C. for 24 hours. The resultant polymer was stirred in dilute HCl solution (5M, 28 mL) for 1 hour, washed with methanol and dried under vacuum at 50° C. for 6 hours.

### Preparation of Polymer F:

Poly(vinylbenzyl chloride) (P-BnCl) polymer (1 g, 5.5 mmol Cl/g, 2.75 mmol) and CH₃CN (20 mL) were stirred in H₂O (1 mL, 55.5 mmol) at 120° C. for 48 hours. The polymer was washed with H₂O, acetone, filtered and dried under vacuum at 50° C. for 24 hours. After synthesis, the catalysts were stirred in dilute HCl solution (5M, 28 mL) for 1 hour, washed with MeOH, filtered and dried under vacuum at 50° C. for 6 hours.

### Preparation of Polymer G:

Poly(vinylbenzyl chloride) (P-BnCl) polymer (1 g, 5.5 mmol Cl/g, 2.75 mmol) and diethylamine (27.5 mmol) were stirred in acetonitrile (33 mL) at 120° C. for hours. The polymer was washed with water and acetone and filtered and dried under vacuum at 50° C. for 24 hours. The resultant polymer was stirred in dilute HCl solution (5M, 28 mL) for 1 hour, washed with methanol and dried under vacuum at 50° C. for 6 hours.

The ammonium loading for each polymer was analyzed by elemental analysis (Table 2).

TABLE 2
Elemental analysis results of polymers

| | C | | | Cl | Total Cl |
|---|---|---|---|---|---|
| Sample | (wt %) | H (wt %) | N (wt %) | (wt %) | (mmol/g) |
| polymer A | 72.76 | 6.37 | 0.76 | 17.72 | 4.99 |
| polymer E | 83.73 | 8.55 | 5.33 | 14.68 | 4.14 |
| polymer F | 72.53 | 7.85 | 7.55 | 21.02 | 5.92 |
| polymer C | 73.45 | 6.28 | 1.00 | 12.26 | 3.45 |
| Polymer C after | 76.71 | 7.04 | 1.04 | 4.19 | 1.18 |
| 12 recycle runs | | | | | |

### Example 3

### Overview of Catalytic Testing

The synthesised polymers were tested for catalytic activity in the conversion of fructose to HMF in isopropanol, as summarised in Table 3.

TABLE 3
Screening of poly-benzylammonium chloride polymers as
catalysts for fructose dehydration in isopropanol.

| | | | Loading | | | |
|---|---|---|---|---|---|---|
| | | | Benzyl- | | | |
| | | | ammonium | Benzyl | | Yield |
| | | Reaction | chloride | chloride | Conver- | A:B |
| Entry | Polymer | condition | (mmol/g) | (mmol/g) | sion | (%) |
| 1 | A | I | 0.54 | 4.45 | 100 | 52:4 |
| 2 | B | I | 0.61 | 4.82 | 100 | 60:4 |
| 3 | C | I | 0.71 | 2.74 | 100 | 60:5 |
| 4 | C | II | 0.71 | 2.74 | 100 | 55:3 |
| 5 | C | III | 0.71 | 2.74 | 100 | 62:4 |
| 6 | D | I | 0.94 | 3.87 | 100 | 61:3 |
| 7 | E | I | 3.8 | 0.34 | 98 | 48:0 |
| 8 | E | II | 3.8 | 0.34 | 97 | 50:1 |
| 9 | E | III | 3.8 | 0.34 | 100 | 52:0 |
| 10 | F | I | 5.39 | 0.53 | 95 | 42:0 |
| 11 | F | II | 5.39 | 0.53 | 94 | 46:0 |
| 12 | F | III | 5.39 | 0.53 | 100 | 50:0 |
| 13 | G | I | 3.5 | 3.86 | 100 | 60:2 |
| 14 | P-BnCl | IV | 0 | 5.50 | 100 | 55:0 |

The scheme below describes the fructose dehydration reaction and the possible products that may form.

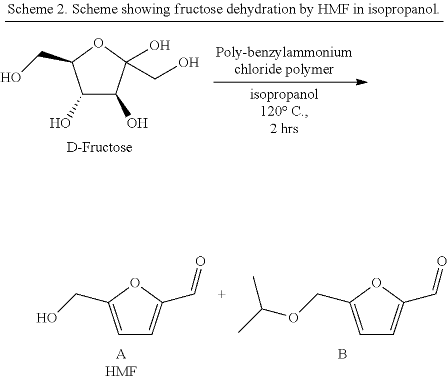

### Reaction Conditions:

All the reactions in Table 3 were performed in the following manner. In a sealed tube equipped with a stirrer bar, fructose (180 mg, 1 mmol) was stirred in isopropanol (5 mL) at 120° C. for 2 hours, following addition of one of the following:

I: 10 mol % polymer based on benzylammonium chloride to fructose
II: 10 mol % polymer based on total chloride to fructose
III: 10 mol % polymer based on benzyl chloride to fructose
IV: 140 mg of polymer
The reaction mixture was cooled to room temperature and filtered. The catalyst, isolated by filtration, was washed with methanol and the combined filtrates were concentrated under reduced pressure to obtain the crude HMF product. For NMR analysis of the composition, mesitylene (0.06 g, 0.5 mmol) was added as an internal standard. Yields were determined by NMR analysis.

The initial testing results showed that poly-benzylammonium polymers generally had good activity for the dehydration of fructose to HMF. All catalysts yielded a mixture of products, namely HMF (major) and HMF-isopropyl ether (minor). Both benzyl ammonium chloride polymers (polymers A to F) and the benzyldiethylammonium analogue (polymer G) were shown to activate the reaction.

Polymers with different ammonium loadings demonstrated different activities. However, no simple linear relationship could be observed. Polymers with an ammonium loading ranging from 10 to 20% of total chlorine were observed to give higher HMF yield as compared to other polymers with lower or higher ammonium loadings.

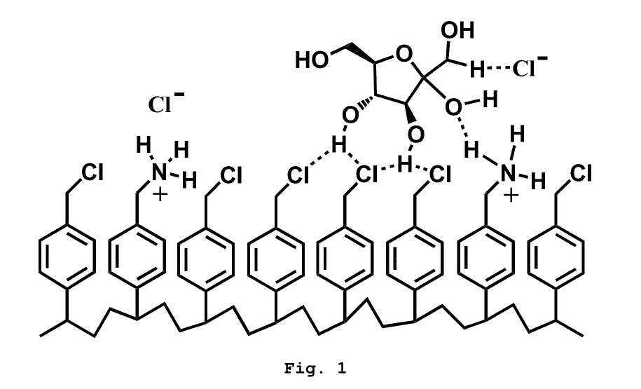

It is thought that both the benzyl chloride groups and the ammonium chloride groups are critical in the catalytic dehydration of fructose. As depicted in FIG. 1, multiple benzyl chloride groups may assist in binding the fructose to the polymer surface via H-bonding with functional groups of fructose, while the HCl dissociated from the ammonium chloride functionality promotes the dehydration process.

Interestingly, Poly(vinylbenzyl chloride) (P-BnCl) itself (Entry 14, Table 3) also showed some catalytic activity. This is thought to be due to the nucleophilic isopropanol substituting benzyl chloride to form benzyl ether and HCl under reaction conditions.

### Example 4

### Reaction Kinetics

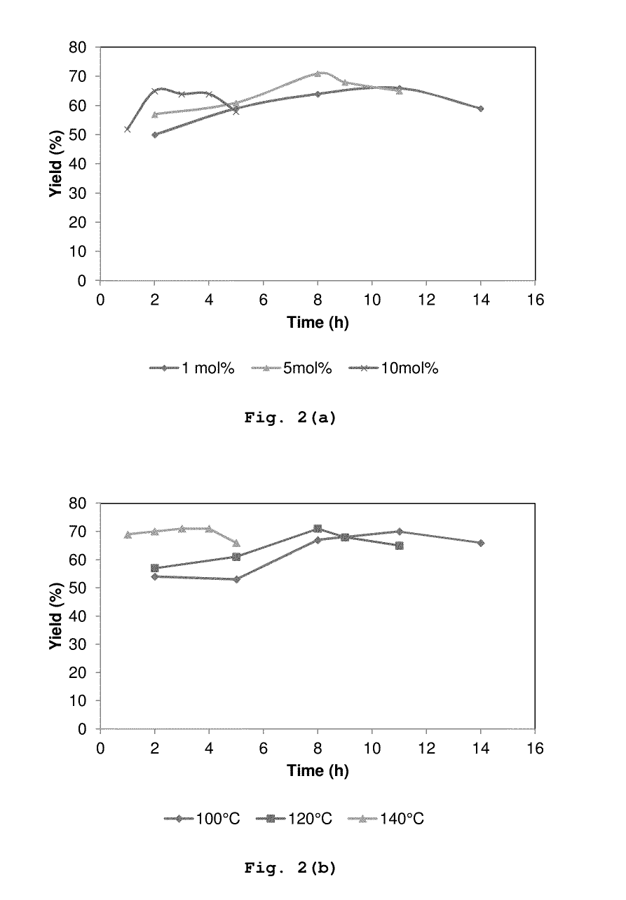

Based on the results observed in Example 3, polymer C with 2.74 mmol/g of benzyl chloride and 0.71 mmol/g of ammonium chloride was selected for further optimization as it gave the best results in Example 3, Table 3. Using the same reaction conditions of Example 3, the effect of catalyst loading on the kinetics of the reaction was investigated by varying the amount of catalyst added. FIG. 2 shows that when catalyst loading was increased from 1 mol % (based on ammonium, chloride to fructose) to 5 mol %, the time for the reaction to reach completion was shortened from 11 hours to 8 hours at 120° C.

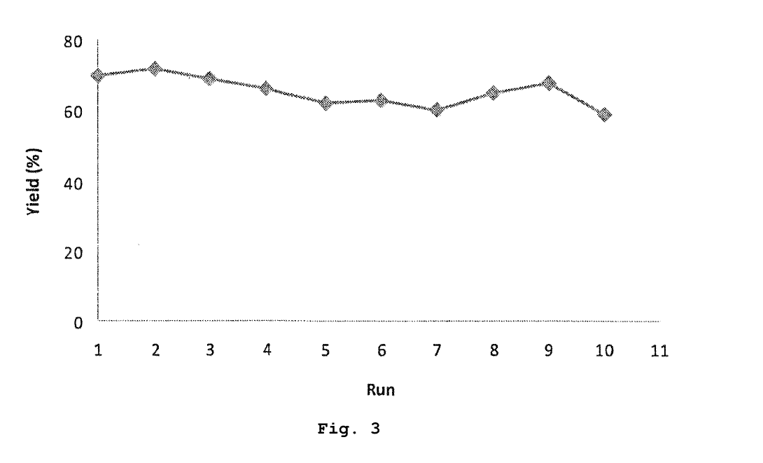

In a similar manner, the effect of changing the temperature on the kinetics of the reaction was investigated. FIG. 3 shows that as the reaction temperature was increased to 140° C., the reaction rate increased and high HMF yield (70%) could be achieved within one hour.

### Example 5

### Recyclability of the Catalytic Polymer

The recyclability of the solid polymer catalysts were evaluated at 140° C. for 3 hours. The recyclability of the polymer was tested by simply washing the polymer with methanol after each round of reaction and drying it under vacuum at 50° C. for 2 hours before using it for another round of reaction. No regeneration steps were required. Each round of reaction was performed with fructose (180 mg) and polymer C (70 mg, 5 mol %) stirred in isopropanol (5 mL) at 140° C. for 3 hours. As shown in FIG. 3, the poly-benzylammonium chloride polymer, as exemplified by polymer C, demonstrated excellent recyclability as a catalyst for this reaction.

The high recyclability is thought to arise from the equilibrium between ammonium and amine/HCl, as discussed in Example 1 and depicted in Scheme 1. It is expected that the catalytic polymer retains its acidic active sites during the reaction cycle, therefore enabling them to be used repeatedly without suffering from decreased activity over multiple use and catalyst deactivation.

### Comparative Example 1

### Comparison with Other Solid Catalysts

Compared to other solid catalysts such as Amberlyst 15 and zeolites, poly-benzylammonium chloride catalysts demonstrated the highest yield as well as recyclability in the conversion of fructose to HMF (Table 3).

TABLE 4
Table comparing the catalytic activity of other solid
catalysts relative to poly-benzylammonium chloride polymers.

| Catalyst | Solvent | Conversion (%) | HMF Yield (%) |
|---|---|---|---|
| H-mordenite | isopropanol | 91 | 17 |
| H-beta | isopropanol | 62 | 2 |
| Amberlyst 15 | isopropanol | 100 | 64 |
| Polymer C | isopropanol | 100 | 73 |
| Polymer C | Biphasic | 85 | 58 |

For Table 4, the conversion was determined using the sugar analyser and the HMF yield was determined based on NMR analysis. For all reactions in Table 4 where the solvent was isopropanol, fructose (180 mg) and the catalyst (70 mg or 5 mol % for Amberlyst 15) in isopropanol (5 mL) were stirred at 140° C. for 3 hours. For the reaction performed in the biphasic solvent, a mixture of fructose (180 mg), NaCl (210 mg), water (0.6 mL), 1-butanol (1.92 mL) and polymer C (70 mg) was stirred at 180° C. for 3 hours. This reaction condition for the biphasic experiment was chosen as it was the optimal condition for the experiment.

In comparison to Amberlyst resin which cannot be used in aqueous/NaCl systems due to the ion exchange effect, the poly-benzylammonium chloride polymers were found to efficiently catalyse the conversion of fructose to HMF in aqueous (NaCl)/organic biphasic solvent systems.

### Comparative Example 2

### Comparison with Other Solid Catalysts

TABLE 5
The comparison of different solid catalysts on fructose
dehydration showing product selectivity.

| | | Conversion | Yield (%)^{b} |
|---|---|---|---|
| Catalyst | Solvent | (%)^{a} | A:B |
| polymer C | Isopropanol | 100 | 71 (60:11) |
| Amberlyst 15 | Isopropanol | 100 | 64 (52:12) |
| Zeolite, H-M | Isopropanol | 91 | 17 (15:2) |
| Zeolite, H-beta | Isopropanol | 62 | 2 (2:0) |
| Zeolite, ZSM-5 | Isopropanol | 74 | 22 (22:0) |
| Zeolite, H-Y | Isopropanol | 98 | 52 (20:32) |
| Sn-beta | Isopropanol | 0 | 0 |
| Al₂O₃ | Isopropanol | 0 | 0 |
| HNb₃O₈ (nanosheet) | Isopropanol | 70 | trace |
| polymer C | DMSO | 100 | 73 (73:0) |
| polymer C | Biphasic^{d} | 85 | 58 (58:0) |

The same reaction conditions as in Comparative Example was used for all reactions in Comparative Example 2.

The high selectivity, good recyclability and versatility of the poly-benzylammonium chloride polymers make them excellent catalysts for the dehydration reaction of a sugar such as fructose to an optionally substituted furan such as HMF.

### Applications

The disclosed polymer may have applications as a catalyst, ion-exchange resin, in casting, plastics, adhesives and in composites.

The disclosed method for synthesising a polymer may be suitable for industrial-scale synthesis of the polymer, as it is harmless to both living organisms and the environment and may facilitate the manufacture of the polymer in a “green” or sustainable manner.

The disclosed polymer may be useful in catalysing the conversion of a sugar to an optionally substituted furan on an industrial scale.

The disclosed method for making an optionally substituted furan from a sugar in the presence of a catalytic polymer may be useful in industrial-scale manufacture of 5-hydroxymethylfuran, as it is efficient and sustainable, which may lead to energy savings.

The disclosed method for making 5-hydroxymethylfuran may be useful in converting low-value biomass chemicals such as fructose to high-value, industrially useful chemicals such as 5-hydroxymethylfuran.

The 5-hydroxymethylfurfural made by the disclosed method may be useful in the manufacture of biofuels more efficient than bioethanol and precursors for polyesters and polyurethanes.

The disclosed method for synthesising a 5-hydroxymethylfurfural may be suitable for industrial-scale synthesis of the substituted furan, as it is efficient, proceeds in mild reaction conditions, under ambient temperature and pressure, in a variety of common and non-harmful solvents, may have a high conversion and yield and may not require high energy input for the reaction to proceed.

The disclosed method for synthesising a 5-hydroxymethylfurfural maybe a “green” or sustainable method of synthesis, as the method and the reagents used therein are harmless to both living organisms and the environment.

The disclosed method for reusing the catalyst may be useful for extended and repeated use of the catalyst in the industrial-scale synthesis of 5-hydroxymethylfurfural.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A polymer comprising an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

2. The polymer according to claim 1, wherein the number ratio of first aromatic ring types represented as “n” to second aromatic ring types represented as “m” is selected to catalyse the dehydration of sugar.

3. The polymer according to claim 1, wherein the mole ratio of n:m is in the range of 20:1 to 1:20 or 10:1 to 1:10.

4. (canceled)

5. The polymer according to claim 1, wherein the aliphatic backbone is a straight chain aliphatic polymer or a saturated straight chain aliphatic polymer.

6. (canceled)

7. The polymer according to claim 1, wherein the aromatic ring is benzene.

8. The polymer according to claim 1, wherein the alkyl of the alkyl halide group of the first aromatic ring type is selected from the group consisting of methyl, optionally substituted ethyl, optionally substituted propyl, optionally substituted butyl, optionally substituted pentyl and any isomers thereof.

9. The polymer according to claim 1, wherein the halide of the alkyl halide group of the first aromatic ring type is selected from the group consisting of fluoride, chloride, bromide and iodide.

10. The polymer according to claim 1, wherein the alkyl halide group of the first aromatic ring type is methyl chloride.

11. The polymer according to claim 1, wherein the first aromatic ring type that has an alkyl halide group substitution on the aromatic ring is benzyl chloride.

12. The polymer according to claim 1, wherein the optionally substituted ammonium of the optionally substituted ammonium chloride group of the second aromatic ring type is selected from the group consisting of primary ammonium, secondary ammonium, tertiary ammonium and quaternary ammonium or is an optionally substituted ammonium chloride.

13. (canceled)

14. The polymer according to claim 1, wherein the second aromatic ring type that has an optionally substituted ammonium halide group substitution on the aromatic ring is optionally substituted benzylammonium chloride.

15. The polymer according to claim 14, wherein the optionally substituted benzylammonium chloride is selected from the group consisting of benzylammonium chloride, benzylurea chloride and diethylbenzylammonium chloride.

16. (canceled)

17. A method for making an optionally substituted furan, the method comprising the step of subjecting a sugar to a dehydration reaction in the presence of a catalyst comprising a polymer having an aliphatic backbone having a plurality of aromatic rings bonded thereon, said plurality of aromatic rings comprising a first aromatic ring type that has an alkyl halide group substitution on the aromatic ring and a second aromatic ring type that has an optionally substituted ammonium halide group substitution on the aromatic ring.

18. The method according to claim 17, wherein the sugar is a monosaccharide or fructose.

19. (canceled)

20. The method according to claim 17, wherein the optionally substituted furan comprises an aldehyde and an alcohol or 5-hydroxymethylfurfural (HMF).

21. (canceled)

22. The method according to claim 17, wherein the dehydration reaction is performed at a temperature in the range of 80° C. to 220° C. or 120° C. to 180° C. at atmospheric pressure and for a time period in the range of 0.1 hours to 20 hours.

23. (canceled)

24. (canceled)

25. (canceled)

26. The method according to claim 17, wherein the catalyst is present in an amount in the range of 0.1 mol % to 30 mol % or 10 mol % to 20 mol % based on optionally substituted ammonium chloride to fructose.

27. (canceled)

28. The method according to claim 17, wherein the dehydration reaction further comprises a solvent selected from the group consisting of water, isopropanol, 1-butanol, N,N-dimethylformamide (DMF), dimethylsulfoxide (DMSO), acetone, acetonitrile, tetrahydrofuran (THF) and any mixtures thereof.

29. (canceled)

30. (canceled)

31. A method for synthesizing a polymer, the method comprising the step of mixing:
(a) a polymer comprising an aliphatic backbone having a plurality of aromatic rings that has an alkyl halide group substitution on the aromatic rings; and
(b) an optionally substituted amine.
